(19) United States
(12) Patent Application Publication
(10) Pub. No.: US 2023/0414361 A1
(43) Pub. Date: Dec. 28, 2023

(54) DEVICES AND METHODS FOR MECHANICALLY INDUCED VENTRICULAR GROWTH IN SINGLE VENTRICLE PATIENTS

(71) Applicant: THE BOARD OF TRUSTEES OF THE LELAND STANFORD JUNIOR UNIVERSITY, Stanford, CA (US)

(72) Inventors: Kyung Won Han, Stanford, CA (US); Alison Marsden, Stanford, CA (US); Mark R. Cutkosky, Palo Alto, CA (US); Ali Kight, Palo Alto, CA (US); Ileana Pirozzi, Palo Alto, CA (US); Michael Ma, Palo Alto, CA (US)

(21) Appl. No.: 18/242,488

(22) Filed: Sep. 5, 2023

Related U.S. Application Data
(63) Continuation of application No. PCT/US22/19393, filed on Mar. 8, 2022.
(60) Provisional application No. 63/158,317, filed on Mar. 8, 2021.
(60) Provisional application No. 63/272,647, filed on Oct. 27, 2021.

(51) Int. Cl. A61F 2/24

(52) U.S. Cl. CPC A61F 2/2481; A61F 2210/0057; A61F 2230/0004; A61F 2220/0075; A61F 2210/0004; A61F 2250/0031; A61F 2220/0016

(57) ABSTRACT

Devices are provided for mechanically induced ventricular growth in a single ventricle patient that include a body comprising a plurality of springs coupled together to define an open upper end and a closed lower end, wherein the springs surround an interior region of the body sized to receive a portion of a patient's heart. The device may be secured over a portion of a patient's heart, e.g., overlying the left ventricular region, and the bias of the springs may apply strain to the myocardium of the heart to induce ventricular chamber growth.

### RELATED APPLICATION DATA

The present application is a continuation of co-pending International Application No. PCT/US2022/019393, filed Mar. 8, 2022, which claims benefit of U.S. provisional application Ser. No. 63/158,317, filed Mar. 8, 2021, and 63/272,647, filed Oct. 27, 2021 the entire disclosures of which are expressly incorporated by reference.

### STATEMENT REGARDING FEDERALLY SPONSORED RESEARCH AND DEVELOPMENT

None.

### TECHNICAL FIELD

The present invention relates to medical devices and, more particularly, to devices for mechanically inducing ventricular growth, e.g., in single ventricle patients, and to methods for implanting and using such devices.

### BACKGROUND

Hypoplastic left heart syndrome (“HLHS”) is a birth defect that affects normal blood flow through the heart. As a baby develops during pregnancy, the left side of the heart does not form correctly, which may result in a hypoplastic left ventricle, e.g., as shown in FIG. 1B, as compared to a normal heart, e.g., as shown in FIG. 1A. Existing treatments for single-ventricle patients are palliative in nature. Avenues for biventricular restoration have largely been limited to mechanical circulatory support (MCS) devices and cardiac transplantation. MCS devices pose a high risk for thrombolytic events, and cardiac transplantation is limited by the amount of donor hearts.

Current surgical palliation for single ventricle physiology involves bypassing the hypoplastic ventricle to convert the circulation into a one-pump system, e.g., as shown in FIG. 1C. Within this paradigm, most current research in myocardial biology and surgical methods is directed towards maintaining the health and function of the systemic single ventricle for as long as possible. Thus, the current treatment of complex single ventricle patients is primarily palliative in nature, and less attention has been paid to strategies for restoring biventricular or one-and-a-half ventricle circulation towards a true functional cure. Avenues for biventricular restoration have largely been limited to mechanical circulatory support devices and cardiac transplantation, with less attention paid to technologies aimed at regrowing or salvaging the existing ventricle.

Therefore, devices and methods for treating patients with hypoplastic ventricles would be useful.

### SUMMARY

The present application is directed to medical devices and, more particularly, to devices for mechanically inducing ventricular growth in single ventricle patients, and to methods for implanting and using such devices. The devices and methods herein may induce favorable growth, e.g., by exerting mechanical stimuli on the myocardial tissue of a hypoplastic ventricle to partially or fully restore size and function of a patient's heart.

For example, the devices disclosed herein may induce favorable growth by exerting selective, controlled mechanical stimuli on the myocardial tissue of the left hypoplastic ventricle to partially or fully restore size and function. It is known that mechanical forces contribute to tissue growth and remodeling in the cardiovascular system. In the case of the hypoplastic heart, this device-based intervention aims to promote volumetric growth through controlled mechanical stimuli (e.g., stretch) to increase the capacity of the hypoplastic ventricle. For example, the devices may be mechanically programmed to exert about fifteen percent (15%) stretch to the cardiac tissue, an amount that has been empirically determined to induce growth but not injure the tissue.

In one example, the device may be implanted beginning at four-to-six (4-6) months of age and remain for several months. The device may be designed to be attached to the epicardium of the hypoplastic ventricle to avoid interference with internal structures or blood flow. In this period, the device may increase the ventricular end-diastolic volume of neonates with a hypoplastic ventricle by two to three times. The device may be programmed to expand over this period synchronously with the growth of the native heart in order to maintain the necessary degree of stretch (e.g., about 15%) to stimulate continued growth.

Alternatively, the device may be implanted during the Norwood procedure, a few days after birth, and removed during the Glenn procedure, at approximately four months of age. In this period, the device is intended to increase the left ventricular end-diastolic volume of neonates with borderline hypoplastic left heart syndrome (HLHS) by approximately three times. The device may be attached to the epicardium of the left ventricle to avoid interference with internal structures or blood flow. The device may be compatible with cardiac contraction through the use of compliant materials and biomimetic design methods. In one example, the device is programmed to expand over four months synchronously with the growth of the native heart in order to maintain the necessary degree of stretch (e.g., about 15%) to stimulate continued growth.

In accordance with one example, a device is provided for mechanically induced ventricular and/or other cardiac growth that includes a body comprising a plurality of spring members coupled together to define an open upper end and a lower end, wherein the spring members surround an interior region of the body sized to receive a portion of a patient's heart and are configured to apply strain to the epicardium of the heart to induce ventricular growth.

In accordance with another example, a device is provided for mechanically induced ventricular growth that includes a body comprising an open upper end and a closed lower end, the body formed by a plurality of spring members coupled together at their opposite ends in an array to define open regions between the spring members through the body, wherein the spring members surround an interior region of the body sized to receive a portion of a patient's heart and are configured to apply strain to the epicardium of the heart to induce ventricular growth. In one example, the array comprises first and second sets of spring members extending orthogonally relative to one another and interconnected at their opposite ends to define the open regions.

In accordance with still another example, a device is provided for mechanically induced ventricular growth that includes a body comprising an open upper end and a closed lower end, the body formed by a plurality of first spring members coupled together at their opposite ends and extending between the upper and lower ends, and a plurality of second spring members coupled together at their opposite ends and extending circumferentially around the body between the upper and lower ends, the first and second spring members interconnected to define open regions through the body, wherein the first and second spring members surround an interior region of the body sized to receive a portion of a patient's heart and are configured to apply strain to the epicardium of the heart to induce ventricular growth.

In accordance with yet another example, a device is provided for mechanically induced ventricular growth that includes a body comprising an open upper end and a closed lower end, the body formed by a plurality of spring members coupled together at interconnection locations in an array to define open regions between the spring members through the body; wherein the spring members surround an interior region of the body sized to receive a portion of a patient's heart and are configured to apply strain to the epicardium of the heart to induce ventricular growth; and a plurality of engagement features extending from an inner surface of the body at the interconnection locations.

In accordance with still another example, a method is provided for making a device for mechanically induced ventricular growth that includes providing a plurality of spring members, each spring member comprising a nonlinear region extending along an axis between opposite ends of the spring member; interconnecting the ends of the spring members to define a body, wherein the spring members surround an interior region of the body sized to receive a portion of a patient's heart and are configured to apply strain to the epicardium of the heart to induce ventricular growth.

In accordance with another example, a method is provided for mechanically induced ventricular growth that includes providing a stretch device including an arrangement of spring members coupled together to define a body including an open upper end and a closed lower end surrounding an interior region; positioning a portion of the patient's heart in the interior region of the stretch device; securing the stretch device to the epicardium of the heart; and allowing the bias of the spring members to apply strain to the myocardium of the heart to induce ventricular chamber growth.

Other aspects and features of the present invention will become apparent from consideration of the following description taken in conjunction with the accompanying drawings.

### BRIEF DESCRIPTION OF THE DRAWINGS

The invention is best understood from the following detailed description when read in conjunction with the accompanying drawings. It is emphasized that, according to common practice, the various features and design elements of the drawings are not to-scale. On the contrary, the dimensions of the various features and design elements are arbitrarily expanded or reduced for clarity. Included in the drawings are the following figures.

FIGS. 1A-1C show cross-sections of examples of a normal heart, a heart with hypoplastic left heart syndrome, and a heart with HLHS post-surgery, respectively.

FIG. 2A shows an example of a stretch device attached to the epicardium of a patient's heart that applies strain to the epicardium to induce growth.

FIG. 2B shows the stretch device of FIG. 2A growing as the heart grows to maintain appropriate strain on the epicardium.

FIGS. 3A and 3B show additional examples of stretch devices that may be attached to the epicardium of a patient's heart including an integral arrangement of springs configured to apply strain to the epicardium.

FIG. 3C is a detail showing an example of a spring that may be included in the devices of FIGS. 3A and 3B.

FIG. 3D is a detail showing another example of a spring including constraints to limit elongation of the spring that may be included in the devices of FIGS. 3A and 3B.

FIG. 4A shows another example of a stretch device attached to the epicardium of a heart and including constraints, e.g., sutures, limiting expansion of the device to limit expansion of integral springs included in the device.

FIG. 4B shows the stretch device of FIG. 4A with some of the constraints being cut to release some of the springs to apply further strain to the epicardium.

FIG. 5A shows another example of a stretch device attached to the epicardium of a heart including an integral arrangement of springs and microneedles configured to apply strain to the epicardium.

FIG. 5B is a detail showing an example of a spring including a plurality of microneedles that may be included in a stretch device, such as that shown in FIG. 5A.

FIG. 5C is a cross-sectional view of the spring of FIG. 5B showing the microneedles contacting adjacent tissue.

FIGS. 6A and 6B are details showing examples of microneedles that may be included in a stretch device.

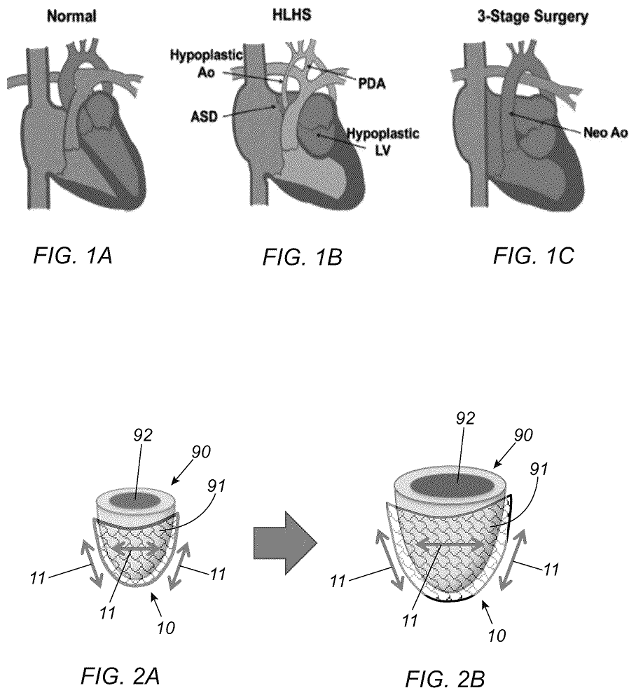

### DETAILED DESCRIPTION

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Turning to the drawings, FIGS. 2A and 2B show an example of a device 10 for mechanically inducing ventricular growth in a single ventricle patient, namely a myocardium stretch device sized to be received over at least a portion over a patient's heart 90. As described further herein, such devices 10 may induce favorable growth, e.g., by exerting mechanical stimuli on the myocardial tissue of the hypoplastic ventricle to partially or fully restore size and function of the patient's heart 90. For example, as shown in FIG. 2A, the device 10 may be implanted to the epicardium 91 over at least the left ventricle 92 of the heart 90, e.g., to avoid interference with internal structures or blood flow within the heart 90, i.e. to induce ventricular chamber growth. As the left ventricle 92 grows, the device 90 may grow and continue to apply strain (as represented by arrows 11) to the epicardium 91, e.g., as shown in FIG. 2B.

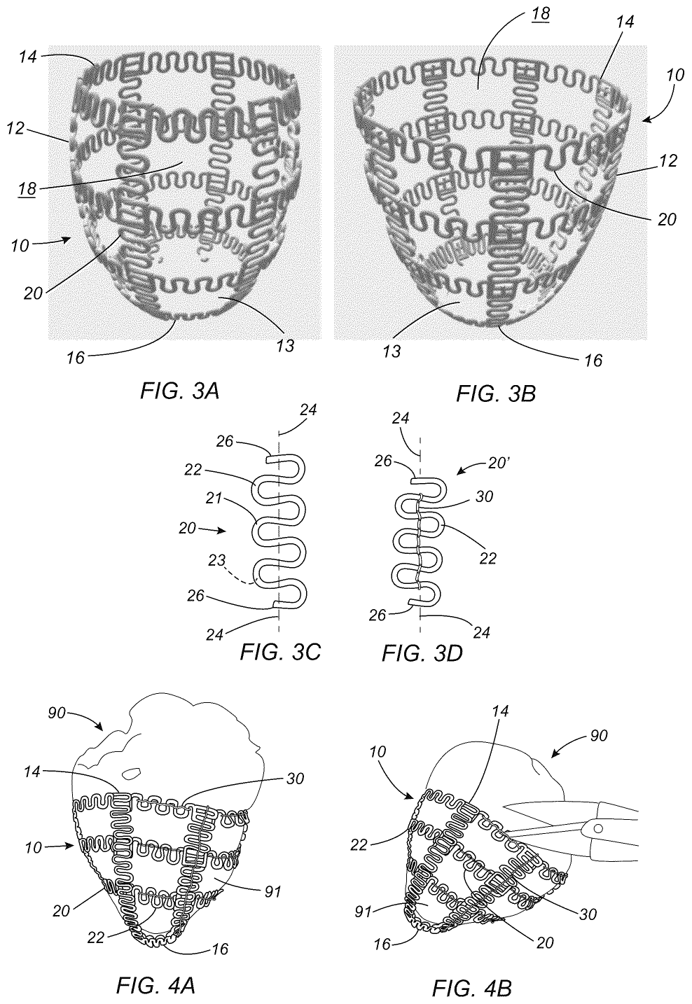

Turning to FIGS. 3A-3C, an exemplary device 10 is shown that includes a plurality of springs 20 coupled together to define a body 12 including an open upper end 14, a closed and/or rounded lower end 16, a plurality of open regions 13 defined by the springs 20. The springs 20 may surround an interior region 18 of the body 12 that is sized to receive a portion of a patient's heart 90, e.g., as shown in FIGS. 4A and 4B, and may be configured to apply strain to the epicardium 91 of the heart 90, e.g., to induce ventricular growth as described elsewhere herein. In the example shown in FIG. 3A, in its relaxed state, the body 12 may define a partial ovoid or other three-dimensional shape, e.g., such that the body 12 expands partially from the upper end 14 before tapering down to the lower end 16. Alternatively, as shown in FIG. 3B, the body 12 may define a generally conical shape tapering from the upper end 14 inwardly towards the lower end 16.

The body 12 may be sized such that the lower end 16 surrounds and engages the apex 94 of the heart 90, e.g., as shown in FIG. 4A, and the upper end 14 is positioned over the epicardium 91 surrounding and/or above the left ventricle (not shown) within the heart 90. The body 12 may be biased to expand circumferentially and/or otherwise to increase in size once implanted, e.g., to continue to apply strain as the heart 90 grows. Optionally, the body 12 may include one or more features that may be used after implantation to accommodate growth of the heart 90 while continuing to apply strain, e.g., one or more constraints, microneedles, and the like, as described further elsewhere herein.

In one example, the springs 20 may be integrally formed together to define the body 12 with open regions 13 between the springs 20, e.g., such that the springs 20 define the entirety of the body 12. For example, the device 10 may be created, e.g. by molding, casting, 3D printing, and the like, to provide an interconnected array of springs 20. Alternatively, a solid-walled body defining the upper and lower ends 14, 16 may be formed, e.g., by molding, casting, 3D printing, and the like, and then the open regions 13 and resulting springs 20 may be formed by removing material, e.g., by laser cutting, machining, etching, and the like. Alternatively, the springs 20 may be formed separately, e.g., individually or in desired linear arrays or other sets, which may be attached together, e.g., at their ends by one or more of bonding with adhesive, laser welding, fusing, suturing, and the like, to provide the body 12.

The body 12 may be formed from one or more biocompatible materials, e.g., elastomeric material, such as silicone, polylactic acid (PLA), epoxy, Nitinol, or other elastic metals, and the like, that provides the desired strain characteristics to the contacted epicardium. For example, the springs 20 defining the entire body 12 may be formed entirely from elastomeric material. Alternatively, additional materials may be embedded in or otherwise attached to the springs 12 to provide desired mechanical expansion properties. For example, elastic elements, e.g., elastic or superelastic wires formed from Nitinol or other metal, plastic, or composite materials (not shown) may be embedded within the loops 22 between the ends 26 of the springs 20 to enhance or otherwise modify the mechanical properties of the springs 20.

Turning to FIG. 3C, an example of a spring 20 is shown that may be included in the device 10. As shown, the spring 20 is an elongate sinuous element including a zigzag or other nonlinear shape, e.g., including alternating loops 22, extending along a longitudinal axis 24 between opposite ends 26 of the springs 20. The loops 22 and ends 26 may lie within a plane extending along the axis 24, e.g., thereby defining an outer surface 21 and an inner surface 23, which may be substantially flat or otherwise shaped to enhance engagement with tissue contacted by the inner surface 23. The ends 26 of the spring 20 may be coupled together with other springs (not shown, see, e.g., FIGS. 3A and 3B) with an initial compression between the ends 26 of the springs 20, such that the springs 20 are biased to increase in length along the axis 26. For example, if the body 12 is formed from individual spring arrays that are attached together, the springs 20 may be compressed axially, e.g., compressing the loops 22 closer to one another from their relaxed state, to generate initial potential energy before the springs 20 are attached together that biases the springs 20 to elongate axially to apply strain to the contacted tissue.

The resulting array of springs 20 may be configured to apply strain in multiple directions along the epicardium 91 of the heart 90, e.g., both vertically and horizontally along the surface of the heart 90 or in other orthogonal arrangements. For example, at least some of the springs 20 may be arranged in generally horizontal bands around the body 12 to surround the heart 90 and/or some of the springs 20 may be arranged generally vertically, e.g., extending at least partially between the upper and lower ends 14, 16 of the body 12. Alternative arrangements of springs and/or other biasing mechanisms for applying strain are described elsewhere herein and disclosed in provisional application Ser. No. 63/158,317, incorporated by reference herein.

Optionally, as shown in FIG. 3D, the spring 20 may include constraints, e.g., one or more sutures or other filaments 30, configured to limit expansion along the axis 26. For example, one or more sutures 30 may be wound, woven, looped, or otherwise extended between one or more adjacent loops 22 of the spring 20 to compress the loops 22 along the axis 26. Once constrained, the loops 22 may store potential energy that may bias the springs 20 to elongate once released. As desired, one or more lengths of the suture(s) 30 may be cut, e.g., to release one or more of the loops 22 to apply additional axial bias of the spring 20. For example, a single suture may be wound around all of the loops 22 of the spring 20 such that a single cut may release all of the loops 22, thereby generated an axial bias as the loops 22 try to expand. Alternatively, multiple suture lengths may be secured between adjacent loops 22 such that a desired number of suture lengths may be cut to release one or more of the loops 22 of the spring 20, e.g., if a more gradual increase in strain is desired. In a further alternative, the suture (s) 30 may be bioabsorbable such that the suture(s) may dissolve over time in a desired manner to release one or more of the loops 22.

For example, as shown in FIGS. 4A and 4B, several of the springs 20 may include one or more bioabsorbable sutures 30 configured to limit expansion of the respective springs 20 until they dissolve. As shown in FIG. 4A, the device 10 may be implanted to a heart 90 with sutures constraining at least some of the springs 20. The unconstrained springs 20 may initially apply a desired strain to the epicardium 91. As the heart 90 grows, one or more of the sutures 30 may dissolve and release the constrained loops 22 of the springs 20 over time, e.g., as shown in FIG. 4B, thereby applying additional strain to the epicardium 91, e.g., as time passes and the heart 90 grows. Alternatively, if desired or if the sutures 30 are not bioabsorbable, one or more sutures 30 may be cut to release one or more loops 22 and/or springs 20 and enhance the strain applied to the myocardium, e.g., as shown in FIG. 4B.

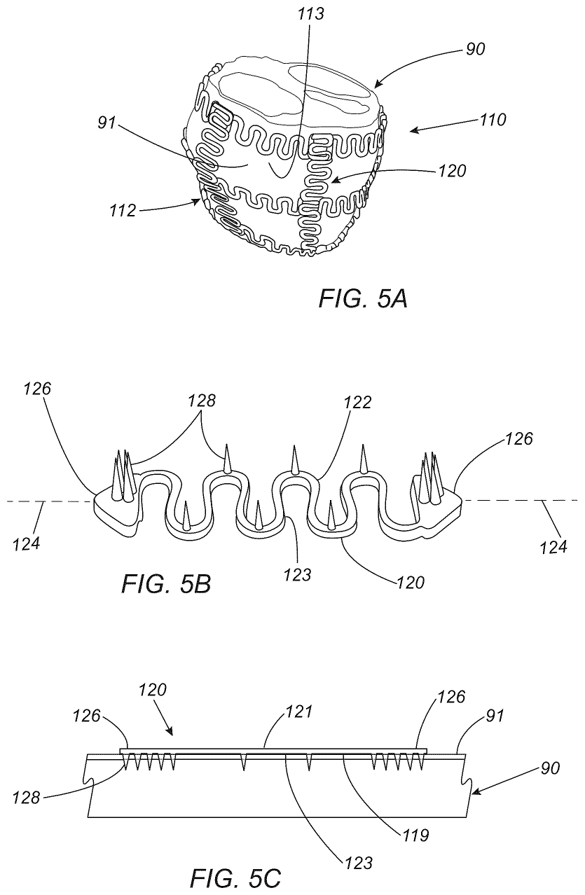

Turning to FIGS. 5A-5C, another device 110 (which may be generally similar to any of the other devices herein) is shown that includes one or more microneedles or other engagement features 128, e.g., extending from an inner surface 119 of the body 112 inwardly to contact tissue received within the interior region 118, e.g., into or against the epicardium 91 of a heart 90, e.g., as shown in FIG. 5C. The microneedles 128 may be integrally formed with the springs 20, e.g., by molding, casting, machining, 3D printing, and the like from the same material, or may be formed separately and attached to the springs 20 or to the finished body 112, as desired. In one example, the microneedles may be formed from atraumatic materials configured to engage contacted tissue without damaging tissue, e.g., elastomeric material, and have lengths from their bases to their tips between about 0.1 and ten millimeters (0.1-10 mm), or about 0.1 to one millimeter (0.1-1.0 mm). Alternatively, the microneedles may be sufficiently rigid and/or long to penetrate into and/or through the myocardium of the heart 90, which may enhance engagement and/or applying strain to the heart 90.

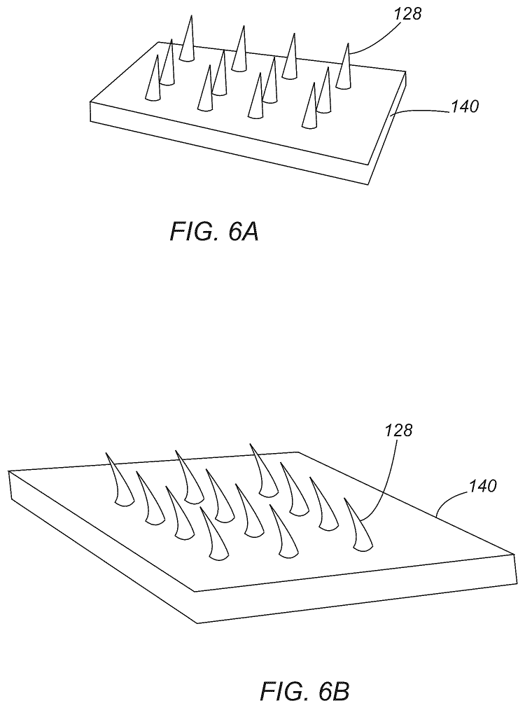

FIGS. 6A and 6B show examples of a set of microneedles 128 formed on a pad 140 that may be permanently attached at desired locations on springs 120 of the body 112. As shown in FIG. 6A, the microneedles 128 may be substantially straight and may extend substantially perpendicular to the surface of the pad 140. Alternatively, as shown in FIG. 6B, the microneedles 128 may curve and/or extend diagonally from the surface, e.g., to provide a primary direction for engagement and/or strain propagation.

For example, as shown in FIG. 5B, pads 140 may be attached at locations where the springs 120 are coupled to one another, e.g., to the inner surface 123 under each end 126 of the spring 120 shown. The microneedles 128 on the pads 140 may secure the ends 124 of the springs 120, e.g., 1) for anchoring the spring-based device to the epicardium 91 and/or 2) for propagating the mechanical stretch through the myocardium of the heart 90 to enhance ventricular growth, e.g., while minimizing damage to tissue. In the example shown in FIG. 5B, a set of microneedles 128 (e.g., on pads 140) may be provided at each of the locations where the ends 126 of the springs 120 are coupled together, which may enhance engagement of the device 110 while allowing the loops 122 of the springs 120 between the ends 126 to apply a potential force, e.g., based on their longitudinal bias, thereby applying strain to the tissue engaged by the microneedles 128. In addition or alternatively, microneedles 128 may be attached directly (or provided on pads attached) to the inner surface 123 under one or more of the loops 122, e.g., to enhance attachment to the epicardium 91 and/or propagate stretch through the myocardium of the heart 90, e.g., as shown in FIGS. 5B and 5C.

In one example, if the springs 120 are formed separately and attached together to provide the body 112, a set of microneedles 128 may be provided at each of the ends 126, as shown in FIG. 5B, before the springs 120 are attached together in the desired arrangement. In addition or alternatively, one or more microneedles 128 may be provided along the length of all or some of the springs 120, e.g., as also shown in FIG. 5B, to enhance engagement with tissue. Optionally, the microneedles 128 may be bioabsorbable, e.g., such that, when the device 120 is removed after treating a patient, the microneedles 128 may separate from the device 120 and remain in the tissue, which may be minimize damage to the heart 90 during removal.

During use, a stretch device 10 such as that shown in FIGS. 4A and 4B, may be provided including an arrangement of springs 20 coupled together to define a body 12 including an open upper end 14 and a closed lower end 16. The configuration and/or spring forces of the springs may be customized, if desired, based on the individual anatomy of the patient's heart 90, or one of a standard set of devices may be selected. The device 20 may be positioned over a portion of the patient's heart 90, e.g., overlying at least the left ventricular region. The stretch device 10 may be secured to the epicardium 91 of the heart 90, e.g., using one or more of sutures, adhesives, clips, or other fasteners (not shown). In addition or alternatively, the inner surface 19 of the body 12 may include materials and/or textures that enhance securing the body 12 relative to the endocardium 91.

After implantation, the bias of the springs 20 may apply strain to the myocardium of the heart 90 to induce ventricular chamber growth. After sufficient time, e.g., several months of growth, the device 10 may be removed. Alternatively, the entire body 12 may be formed from bioabsorbable material that may dissolve and be metabolized after a desired time period.

Although the devices and methods herein have been described with particular reference to inducing ventricular growth, e.g., in single ventricle patients born with hypoplastic left heart syndrome, it will be appreciated that the devices and methods may be used to treat other cardiac and/or pediatric cardiology diseases, e.g., to induce ventricular growth and/or other treatment of a patient's heart.

In describing representative examples, the specification may have presented the method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A device for mechanically induced ventricular and/or other cardiac growth in a patient, comprising:
a body comprising a plurality of spring members coupled together to define an open upper end and a lower end,
wherein the spring members surround an interior region of the body sized to receive a portion of a patient's heart and are configured to apply strain to the epicardium of the heart to induce ventricular growth.

2. (canceled)

3. The device of claim 1, wherein the spring members are coupled together at their opposite ends in an array to define open regions between the spring members through the body, and wherein the array comprises first and second sets of spring members extending orthogonally relative to one another and interconnected at their opposite ends to define the open regions.

4. (canceled)

5. The device of claim 1, wherein the spring members are integrally formed in the body.

6. The device of claim 1, wherein the spring members are configured to apply strain in multiple directions along the surface of the heart.

7. The device of claim 1, wherein the spring members comprise a plurality of sinuous springs.

8. The device of claim 7, wherein the sinuous springs comprise a plurality of loops extending along an axis between opposite ends of the respective springs.

9. The device of claim 8, wherein the loops of respective springs lie within a plane such that springs define an inner surface configured to enhance engagement with contacted tissue.

10. The device of claim 7, wherein each spring is configured to provide an elongation force between ends of the spring to apply the strain to the contacted epicardium.

11. The device of claim 7, further comprising constraints limiting elongation of at least some of the springs.

12. The device of claim 11, wherein the constraints comprise sutures.

13. The device of claim 11, wherein the constraints are bioabsorbable.

14. The device of claim 1, wherein at least some of the spring members comprise constraints that store potential energy in the spring members.

15. The device of claim 14, wherein the constraints are bioabsorbable such that, when the constraints dissolve, they release the constrained spring members to apply the stored potential energy to generate additional strain to the epicardium.

16. The device of claim 14, wherein the constraints comprise sutures.

17. The device of claim 14, wherein each of the spring members comprise a plurality of loops extending along an axis between opposite ends of the respective spring members, and wherein the constraints compress at least some of the loops together along the axis to generate the stored potential energy.

18. The device of claim 1, further comprising one or more engagement features extending from the body inwardly to contact tissue received within the interior region.

19. The device of claim 18, wherein the one or more engagement features comprise microneedles extending from an inner surface of the body.

20. The device of claim 19, wherein the microneedles are provided at interconnection locations where ends of adjacent spring members are attached together.

21-30. (canceled)

31. A method for mechanically induced ventricular and/or other cardiac growth in a patient, comprising:
providing a stretch device including an arrangement of spring members coupled together to define a body including an open upper end and a closed lower end surrounding an interior region;
positioning a portion of the patient's heart in the interior region of the stretch device;
securing the stretch device to the epicardium of the heart; and
allowing the bias of the spring members to apply strain to the myocardium of the heart to induce ventricular chamber growth.

32-40. (canceled)

41. A method for making a device for mechanically induced ventricular growth in a patient, comprising:
providing a plurality of spring members, each spring member comprising a nonlinear region extending along an axis between opposite ends of the spring member;
interconnecting the ends of the spring members to define a body, wherein the spring members surround an interior region of the body sized to receive a portion of a patient's heart and are configured to apply strain to the epicardium of the heart to induce ventricular growth.

42-54. (canceled)
